(19) United States
(12) Patent Application Publication
(10) Pub. No.: US 2006/0247233 A1
(43) Pub. Date: Nov. 2, 2006

(54) Thiazoles inhibitors of the alk-5 receptor

(76) Inventors: Nerina Dodic, Les Ulis (FR); Frederic Donche, Les Ulis (FR); Jeanne Francoise Gellibert, Les Ulis (FR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/560,691

(22) PCT Filed: Jun. 14, 2004
(86) PCT No.: PCT/EP04/06425

§ 371 Date: Apr. 13, 2006

(30) Foreign Application Priority Data
Jun. 16, 2003 (GB) 0313914.4

(51) Int. Cl. A61K 31/538; A61K 31/4709; A61K 31/4439; A61K 31/427; C07D 417/14

(52) U.S. Cl. 514/230.5; 514/314; 514/341; 514/370; 544/105; 546/176; 546/269.7; 548/181

(57) ABSTRACT

The invention relates to novel aminothiazole derivatives which are inhibitors of the transforming growth factor, (“TGF”)-β signaling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway

This invention relates to novel aminothiazole derivatives which are inhibitors of the transforming growth factor, (“TGF”)-β signaling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

### BACKGROUND OF THE INVENTION

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided into two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signaling requires both the ALK and type II receptors. Specifically, the type II receptor phosphorylates the GS domain of the type I receptor for TGF-0, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

WO02/062776, WO02/062753 and WO02/062793 (Glaxo Group Limited) disclose substituted aminothiazole compounds, which are useful In the treatment or prophylaxis of disorders characterised by the overexpression of TGF-β. In particular, the compounds are described as TGF-β inhibitors which act at the TGF-β type I (Alk5) receptor level.

WO04/013134 (SmithKline Beecham Corporation) discloses phenylpyridyl substituted aminothiazole compounds, which are useful in the treatment or prophylaxis of disorders characterised by the overexpression of TGF-β. In particular, the compounds are described as TGF-α inhibitors which act at the TGF-β type I (Alk5) receptor level.

WO2004/026863 (Pfizer) discloses novel oxazole and thiazole compounds which are potent inhibitors of transforming growth factor (“TGF”)-β signalling pathway. The compounds are said to be useful in the treatment of various TGF-related disease states including, for example, cancer and fibrotic disease.

Surprisingly, it has now been discovered that a class of novel aminothiazole derivatives function as potent and selective non-peptide inhibitors of ALK5 kinase.

According to a first aspect, the invention provides a compound of formula (I), a pharmaceutically acceptable salt, solvate or derivative thereof: 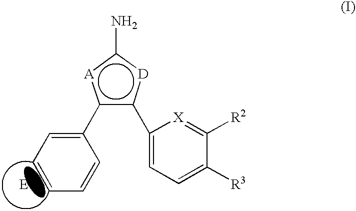
wherein
either A is S and D is N; or A is N and D is S;
ring E is a saturated, unsaturated or aromatic 5 or 6-membered heterocycle which heterocycle in addition to carbon contains one or more ring-heteroatoms Independently selected from nitrogen and oxygen, wherein the heterocycle is optionally substituted on any nitrogen atom where appropriate by one or more groups R^{Ea} independently selected from C₁₋₆alkyl and C₁₋₆alkoxyC₁₋₆alkyl and is optionally substituted on any carbon atom where appropriate by one or more groups R_{Eb} independently selected from oxo, C₁₋₆alkyl, C₁₋₆alkoxyC₁₋₆alkyl, C₁₋₆alkoxy and halo;
X is N or CH;
R² is hydrogen, C₁₋₆alkyl, halo, cyano or perfluoroC₁₋₆alkyl; and
R³ is hydrogen or halo.

Preferably the benzofused ring system including E is selected from the list: benzimidazol-6-yl, benzoxazol-6-yl, benzoxazol-5-yl, 4H-benzo[1,4]oxazin-3-one-6-yl, benzo[1,3]dioxol-5-yl, benzodioxan-6-yl, quinolin-6-yl and benzotriazol-6-yl.

More preferably the benzofused ring system including E is selected from the list: benzimidazol-6-yl, benzoxazol-6-yl, benzoxazol-5-yl, 4H-benzo[1,4]oxazin-3-one-6-yl, benzodioxan-6-yl, and quinolin-6-yl.

Preferably X is N.

Preferably R² is hydrogen, C₁₋₆alkyl, chloro or fluoro. More preferably R² is hydrogen, methyl, chloro or fluoro. More preferably still, R² is methyl.

Preferably R³ is hydrogen.

Preferably, when X is N, R² is methyl. More preferably when X is N and R² is methyl, R³ is H.

It will be appreciated that the present invention is intended to include compounds having any combination of the preferred groups listed hereinbefore.

Compounds of formula (I) which are of special interest as agents useful in the treatment or prophylaxis of disorders characterised by the overexpression of TGF-β are selected from the list:
5-(1-methyl-benzimidazol-6-yl)-4-(6-methylpyridin-2-yl)-1,3-thiazol-2-amine (Example 1);
4-(benzoxazol-6-yl)-5-(6-methyl-pyridin-2-yl)-1,3-hiazol-2-amine (Example 2);
5-(1-ethyl-benzimidazol-6-yl)-4-(6-methylpyridin-2-yl)-1,3-hiazol-2-amine (Example 3);
5-(1-(2-methoxyethyl)-benzimidazol-6-yl)-4-(6-methylpyridin-2-yl)-1,3-thiazol-2-amine (Example 4);
5-[4-methyl-4H-benzo[_{1,4}]oxazin-3-one-6-yl]-4-(6-methylpyridin-2-yl)-1,3-thiazol-2-amine (Example 5);
5-[4-ethyl-4H-benzo[1,4]oxazin-3-one-6-yl]4-(6-methylpyridin-2-yl)-1,3-thiazol-2-amine (Example 6);
4-(benzo[1,3]dioxol-5-yl)-5-(6-methylpyridin-2-yl)-1,3-thiazol-2-amine (Example 7);
4-(benzodioxan-6-yl)-5-(pyridin-2-yl)-1,3-thiazol-2-amine (Example 8);
4-(quinolin-6-yl)-5-(pyridin-2-yl)-1,3-thiazol-2-amine (Example 9);
4-(1-methyl-benzotriazol-6-yl)-5-(pyridin-2-yl)-1,3-thiazol-2-amine (Example 10);
4-(1-methyl-benzimidazol-6-yl)-5-(6-methylpyridin-2-yl)-1,3-thiazol-2-amine (Example 11); and
pharmaceutically acceptable salts, solvates and derivatives thereof.

Compounds which are more preferred are selected from the list:
5-(1-methyl-benzimidazol-6-yl)-4-(6-methylpyridin-2-yl)-1,3-thiazol-2-amine (Example 1);
4-(benzoxazol-6-yl)-5-(6-methyl-pyridin-2-yl)-1,3-thiazol-2-amine (Example 2);
5-(1-ethyl-benzimidazol-6-yl)-4-(6-methylpyridin-2-yl)-1,3-thiazol-2-amine (Example 3);
5-(1-(2-methoxyethyl)-benzimidazol-6-yl)-4-(6-methylpyridin-2-yl)-1,3-thiazol-2-amine (Example 4);
5-[4-methyl-4H-benzo[1,4]oxazin-3-one-6-yl])(6-methylpyridin-2-yl-1,3-thiazol-2-amine (Example 5);
5-[4-ethyl-4H-benzo[1,4]oxazin-3-one-6-yl]-4-(6-methylpyridin-2-yl)-1,3-thiazol-2-amine (Example 6);
4-(benzodioxan-6-yl)-5-(pyridin-2-yl)-1,3-thiazol-2-amine (Example 8);
4-(1-methyl-benzimidazol-6-yl)-5-(6-methylpyridin-2-yl)-1,3-thiazol-2-amine (Example 11); and
pharmaceutically acceptable salts, solvates and derivatives thereof.

The term “C₁₋₆alkyl” as used herein, whether on its own or as part of a group, refers to a straight or branched chain saturated aliphatic hydrocarbon radical of 1 to 6 carbon atoms, unless the chain length is limited thereto, including, but not limited to methyl, ethyl, n-propyl, isopropyl, n-butyl, sec-butyl, isobutyl, tert-butyl, pentyl and hexyl.

The term “alkoxy” as a group or part of a group refers to an alkyl ether radical, wherein the term “alkyl” is defined above. Such alkoxy groups in particular include methoxy, ethoxy, n-propoxy, isopropoxy, n-butoxy, iso-butoxy, sec-butoxy and tert-butoxy.

The term “perfluoroalkyl” as used herein includes compounds such as trifluoromethyl.

The terms “halo” or “halogen” are used interchangeably herein to mean radicals derived from the elements chlorine, fluorine, iodine and bromine.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of posssible substituents, those substituents may be the same or different.

As used herein the term “pharmaceutically acceptable derivative” means any pharmaceutically acceptable salt, solvate, ester or amide, or salt or solvate of such ester or amide, of the compound of formula (I), or any other compound which upon administration to the recipient is capable of providing (directly or indirectly) the a compound of formula (I) or an active metabolite or residue thereof, eg, a prodrug. Preferred pharmaceutically acceptable derivatives according to the invention are any pharmaceutically acceptable salts, solvates or prodrugs.

Suitable pharmaceutically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

Hereinafter, compounds, their pharmaceutically acceptable salts, their solvates and polymorphs, defined in any aspect of the invention (except intermedate compounds in chemical processes) are referred to as “compounds of the invention”.

Compounds of the invention may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Compounds of the invention may be prepared in a variety of ways. In the following reaction schemes and hereafter, unless otherwise stated R¹ to R³ and X are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc . . . (IV), (IVb), (IVc) etc.

Compounds of formula (Ia), i.e. compounds of general formula (I) where A is S and D is N, may be prepared from compounds of formula (II) according to reaction scheme 1 by treating compounds of formula (II) with polymer supported pyridinium perbromide in a suitable solvent such as tetrahydrofuran or dicloromethane at room temperature followed by treatment with thiourea in a suitable solvent such as ethanol at elevated temperature.

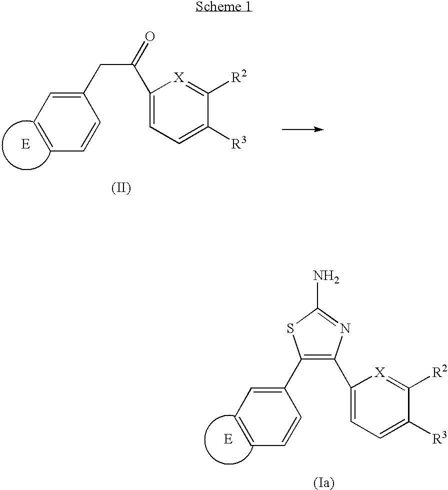

Compounds of formula (Ib), i.e. compounds of general formula (I) where A is N and D is S, may be prepared from compounds of formula (III) according to reaction scheme 2 using analogous reaction conditions to those described for reaction scheme 1.

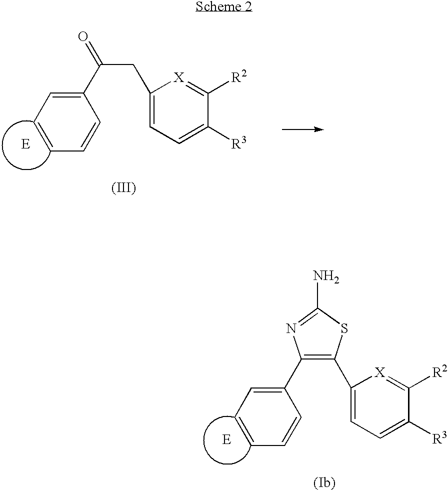

Alternatively compounds of general formula (I) may be prepared by formation of ring E as the final step. For instance, compounds of formula (Ic), i.e. compounds of general formula (I) where the benzofused ring system including E is benzimidazol-6-yl may be prepared according to reaction scheme 3. Preferred reaction conditions comprise reaction with iron in acetic acid at elevated temperatures followed by treatment with trimethylorthoformate at elevated temperature.

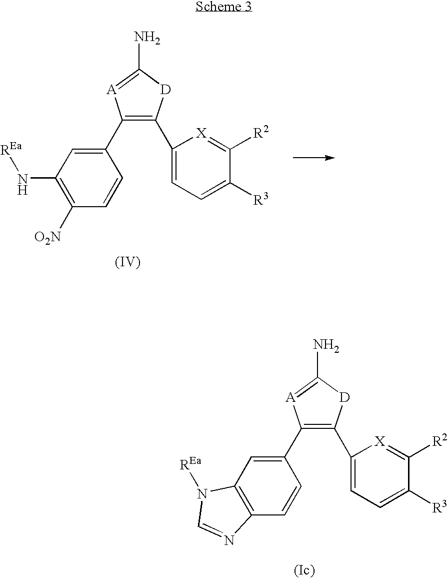

In addition, compounds of formula (Id), i.e. compounds of general formula (I) where the benzofused ring system including E is a benzoxazol-6-yl, may be prepared according to reaction scheme 4. Preferred reaction conditions comprise reaction with iron in acetic acid at elevated temperatures followed by treatment with a compound of formula (V) in a suitable solvent such as ethanol at elevated temperature.

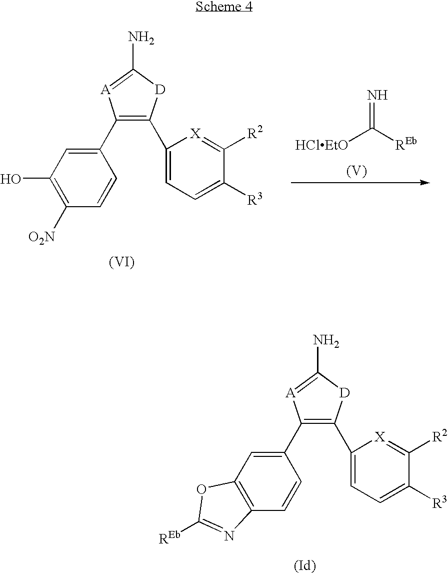

Compounds of formula (II) (see scheme 1) may be prepared according to reaction scheme 5 by reacting aldehydes of formula (VII) with N,P acetals of formula (VIII) followed by hydrolysis of the resulting enamine (see M. Jounet, Tetrahedron Letters, 1998, 39, 1717-1720 and I. W. Davies et al., J. Org. Chem., 2000, 65, 8415-8420). Preferred reaction conditions comprise treatment with a suitable base, such as cesium carbonate or potassium tert-butoxidel in a suitable solvent such as tetrahydrofuran and isopropyl alcohol. The enamine may be hydrolysed with hydrochloric acid.

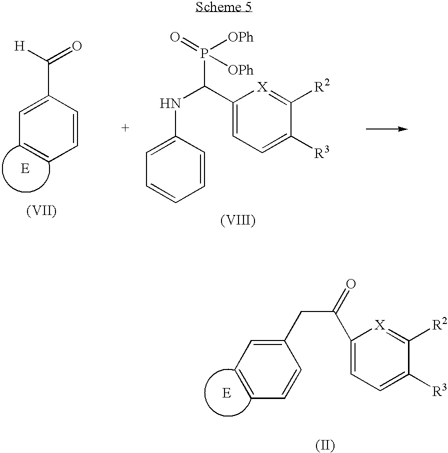

Compounds of formula (III) may be prepared according to reaction scheme 6, by coupling N,P acetals of formula (IX) with compounds of formula (X) under analogous conditions to those described for reaction scheme 5.

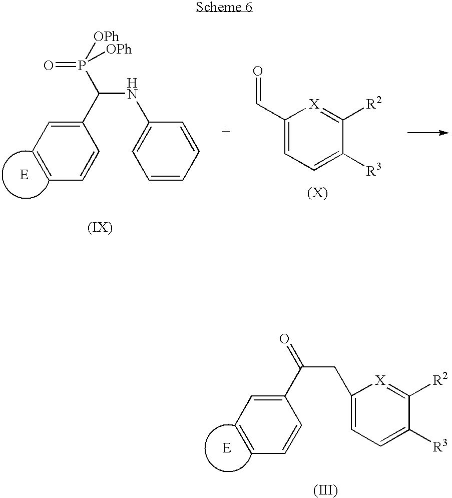

Compounds of formula (IIIa), i.e. compounds of (III) (see scheme 2) where X is N, R² is methyl, R³ is H and where the benzofused ring system including E is a benzo[1,3]dioxol-5-yl, benzodioxan-6-yl, quinolin-6-yl and benzotriazol-6-yl, may be prepared according to reaction scheme 7. Preferred reaction conditions comprise coupling 2,6-lutidine with compounds of formula (XI) in the presence of a suitable base such as n-butyl lithium or sodium bis (trimethylsilyl) amide at between −20° C. to −50° C. in a solvent such as tetrahydrofuran.

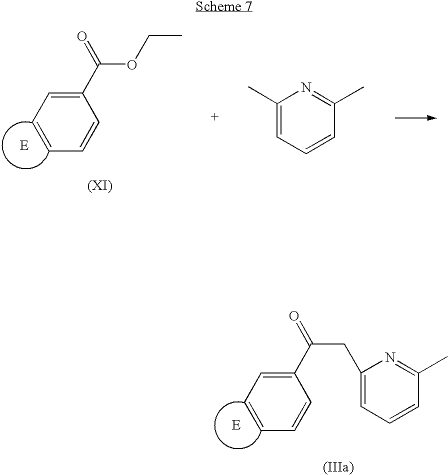

Compounds of formula (VIIa) may be prepared following the general methodology described in Scheme 8.

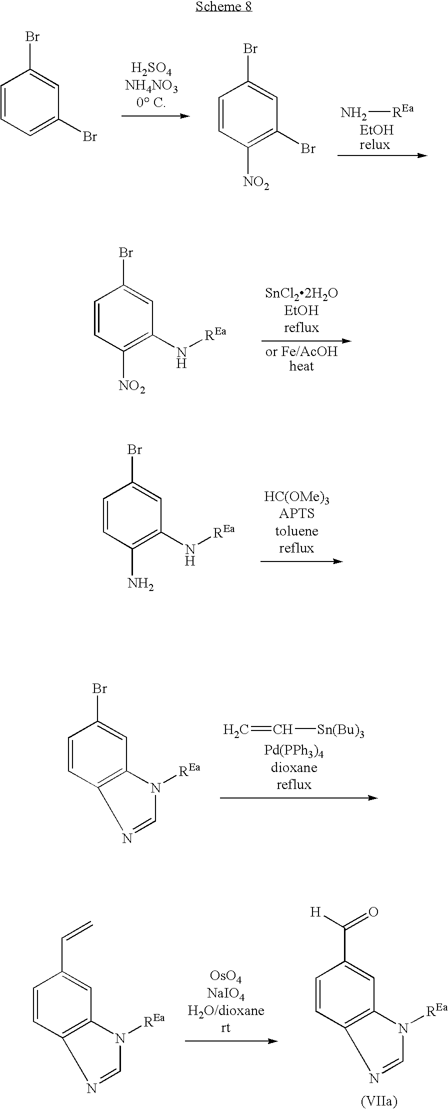

Compounds of formula (VIIb) may be prepared following the general methodology described in Scheme 9.

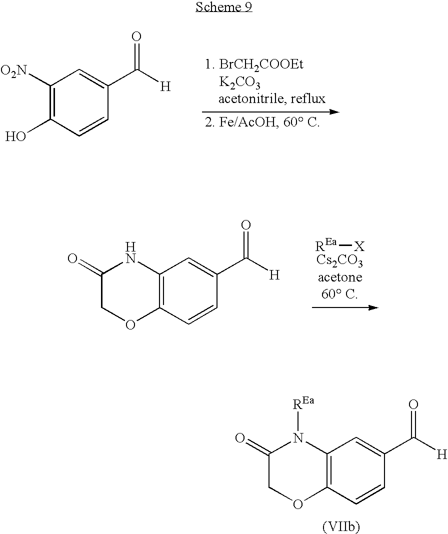

Further details for the preparation of compounds of formula (I) are found in the examples.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Activation of the TGF-β1 axis and expansion of extracellular matrix are early and persistent contributors to the development and progression of chronic renal disease and vascular disease. Border W. A, et al, N. Engl. J. Med., 1994; 331(19), 1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al., Invest. Ophthalmol. Vis. Sci., 1998; 39(11), 1981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19), 1286-92. TGF-β1 is elevated in acute and chronic glomerulonephritis Yoshioka K., et al, Lab. Invest, 1993; 68(2), 154-63, diabetic nephropathy Yamamoto, T., et al, 1993, PNAS 90, 1814-1818., allograft rejection, HIV nephropathy and angiotensin-induced nephropathy Border W. A., et al., N Engl. J. Med., 1994; 331(19), 1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats. Third, TGF-β1 transgenic mice or in vivo transfection of the TGF-β1 gene into normal rat kidneys resulted in the rapid development of glomerulosclerosis. Kopp J. B., et al, Lab. Invest, 1996; 74(6), 991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3), 193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., et al, Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al, Jr., J. Clin. Invest., 1995; 96(6), 2667-75. TGF-β1 was immunolocalized to non-foamy) macrophages in atherosclerotic lesions where active matrix synthesis occurs, suggesting that non-foamy niacrophages may participate in modulating matrix gene expression in atherosclerotic remodeling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signaling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signaling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

Therefore according to a further aspect, the invention provides the use of a compound defined in the first aspect in the preparation of a medicament for treating or preventing a disease or condition mediated by ALK-5 inhibition.

Preferably the disease or condition mediated by ALK-5 inhibition is selected from the list: chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photoaging.

More preferably the disease or condition mediated by ALK-5 inhibition is fibrosis. Preferably kidney fibrosis.

It will be appreciated that references herein to treatment extend to prophylaxis as well as the treatment of established conditions.

Compounds of the present invention may be administered in combination with other therapeutic agents, for example antiviral agents for liver diseases, or in combination with ACE inhibitors or angiotensin II receptor antagonists for kidney diseases.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

No toxicological effects are indicated when a compound of the invention is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The preferred embodiments described for the first aspect extend these further aspects:

i) a pharmaceutical composition comprising a compound of the invention and a pharmaceutically acceptable carrier or diluent;

ii) a compound of the invention for use as a medicament;

iii) a method of treatment or prophylaxis of a disorder selected from chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photoaging, in mammals, which comprises administration to the mammal in need of such treatment, an effective amount of a compound of the invention; and

iv) a combination of a compound of the invention with an ACE inhibitor or an angiotensin II receptor antagonist.

The following non-limiting examples illustrate the present invention.

Abbreviations

AcOH—acetic acid

APTS—p-toluene sulfonic acid

Cs2CO3—cesium carbonate

DCM—dichloromethane

EtOH—ethanol

EtOAc—ethyl acetate

K₂CO₃—potassium carbonate

MeOH—methanol

Na₂SO₄—sodium sulfate

NaI O₄—sodium periodate

Os O₄—osmium tetroxide

THF—tetrahydrofuran

TEA—triethylamine

DME—dimethyoxyethane

Pd₂(dba)₃—tris(dibenzylideneacetone)dipalladium (0)

Binap—2,2′-bis(diphenylphosphino)-1,1′-binaphthyl

iPrOH—isopropanol

NaHMDS—sodium bis(trimethylsilyl)amide

### Intermediate 1: 2,4-dibromo-nitrobenzene

To an iced cold solution of 1,3-dibromobenzene (10 g, 42.3 mmol) in sulfuric acid (200 ml) was added portionwise ammonium nitrate (3.39 g, 42.3 mmol) and the mixture was stirred at 0° C. for 10 minutes and then poured into water. After extraction with CH₂Cl₂, the organic phase was washed with a saturated solution of NaHCO₃, dried over Na₂SO₄ and concentrated under reduced pressure. After trituration with pentane, the title compound was obtained as a pale yellow solid (8 g, 67.2%); NMR H¹ (300 MHz, CDCl₃, ppm) δ: 7.95 (s, 1H), 7.75 (d, 1H), 7.6 (d, 1H).

### Intermediate 2: 4-bromo-2-(methylamino)-nitrobenzene

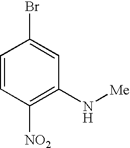

To a solution of intermediate 1 (8 g, 28.5 mmol) in EtOH (200 ml) was added a solution of methylamine 40% in water (200 ml) and the mixture was heated under reflux for 2 hours and then cooled. The resulting precipitate was filtered and dried. The title compound was obtained as an orange solid (5 g, 76%); m.p. 130-132° C.

### Intermediate 3: 4-bromo-2-(ethylamino)-nitrobenzene

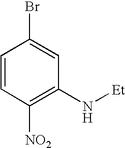

Intermediate 1 (6 g, 28 mmol) and ethylamine (solution 70% in water, 200 ml) were reacted as described for intermediate 2 to afford the title compound as a yellow solid (5 g, 99.9%); NMR H¹ (300 MHz, CDCl₃, ppm) δ: 8.25 (s, 1H), 8.1 (d, 1H), 7.05 (s, 1H), 6.85 (d, 1H), 3.25 (m, 2H), 1.3 (t, 3H).

### Intermediate 4: 4-bromo-2-(2-methoxyethylamino)-nitrobenzene

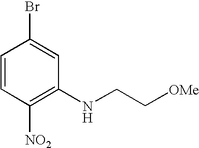

Intermediate 1 (6 g, 28 mmol) was reacted with 2-methoxyethylamine as described for intermediate 2, to afford the title compound as a solid (6 g, 99.9%); NMR H¹ (300 MHz, CDCl₃, ppm) δ: 8.3 (brs, 1H), 8.1 (d, 1H), 7.1 (s, 1H), 6.85 (d, 1H), 3.8 (m, 2H), 3.6 (m, 2H), 3.5 (s, 3H).

### Intermediate 5: 1-methyl-6-bromo-benzimidazole

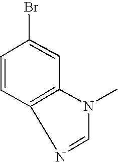

To a solution of intermediate 2 (5 g, 21.6 mmol) in EtOH (200 ml) was added portionwise SnCl₂.2H₂O (9.8 g, 43 mmol) and the mixture was heated under reflux for 4 hours and then concentrated under reduced pressure. The residue was treated with water (200 ml) and NaOH 1N (100 ml). After extraction with CH₂Cl₂, the organic phase was dried over Na₂SO₄ and concentrated under reduced pressure. The residue was dissolved in toluene (50 ml) and trimethylorthoformate (2.6 ml, 24 mmol) and APTS (0.2 g) were added and the mixture was heated under reflux for 2 hours and then concentrated under reduced pressure. The residue was purified by chromatography on silica gel eluting with CH₂Cl₂/MeOH (95/5). The title compound was obtained as a cream powder (2.5 g, 54.74%); m.p. 126-128° C.

### Intermediate 6: 1-ethyl-6-bromo-benzimidazole

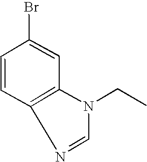

Intermediate 3 (5 g, 22 mmol) was reacted as described for intermediate 5, to afford the title compound as a brown oil (2.3 g, 47.23%); NMR H¹ (300 MHz, CDCl₃, ppm) δ: 8.00 (s, 1H), 7.75 (d, 1H), 7.65 (s, 1H), 7.45 (d, 1H), 4.25 (q, 2H), 1.6 (t, 3H).

### Intermediate 7: 1-(2-methoxyethyl)-6-bromo-benzimidazole

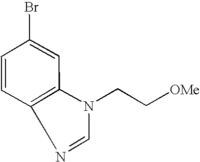

To a solution of intermediate 4 (6 g, 22 mmol) in acetic acid (100 ml) at 60° C. was added portionwise under vigorous stirring, iron (12 g, 220 mmol) and the mixture was heated at 60° C. for 2 hours and then cooled. The reaction mixture was basified by addition of a solution of sodium hydroxide, filtered through celite and the filtrate was extracted with CH₂Cl₂. The organic-phase was dried over Na₂SO₄ and concentrated under reduced pressure. The residue was dissolved in toluene (80 ml) and trimethylorthoformate (3.5 ml, 32 mmol) and APTS (0.6 g) were added and the mixture was heated under reflux overnight and then concentrated under reduced pressure. The residue was purified by chromatography on silica gel eluting with CH₂Cl₂/MeOH (9/1). The title compound was obtained as an oil (6 g, 96.07%); NMR H¹ (300 MHz, CDCl₃, ppm) δ: 8.1 (s, 1H), 7.8 (d, 1H), 7.7 (s, 1H), 7.5 (d, 1H), 4.4 (t, 2H), 3.85 (t, 2H), 3.4 (s, 3H).

### Intermediate 8: 1-methyl-6-vinyl-benzimidazole

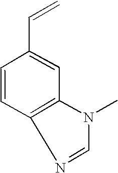

To a solution of intermediate 5 (2.5 g, 11.8 mmol) in dioxane (100 ml) were added tributyl(vinyl)tin (5.2 ml, 18 mmol) and tetrakis(triphenylphosphine)palladium(0) (0.69 g, 0.5 mmol) and the mixture was heated under reflux for 24 hours and then concentrated under reduced pressure. The residue was purified by chromatography on silica gel, eluting with CH₂Cl₂/MeOH (95/5). The title compound was obtained as an oil (1.8 g, 96.1%); NMR H¹ (300 MHz, CDCl₃, ppm) δ: 7.9 (s, 1H), 7.75 (d, 1H), 7.4 (m, 2H), 6.85 (dd, 1H), 5.8 (d, 1H), 5.25 (d, 1H), 3.85 (s, 3H).

### Intermediate 9: 1-ethyl-6-vinyl-benzimidazole

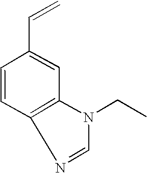

Intermediate 6 (2.3 g, 10 mmol) was reacted as described for intermediate 8 to afford the title compound as an oil (1.5 g, 85.31%); NMR H¹ (300 MHz, CDCl₃, ppm) δ: 7.9 (s, 1H), 7.75 (d, 1H), 7.4 (m, 2H), 6.95 (m, 1H), 5.8 (m, 1H), 5.25 (m, 1H), 4.2 (q, 2H), 1.55 (t, 3H).

### Intermediate 10: 1-(2-methoxyethyl)-6-vinyl-benzimidazole

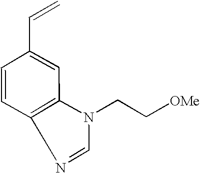

Intermediate 7 (6 g, 23.5 mmol) was reacted as described for intermediate 8 to afford the title compound as an oil (3.5 g, 73.64%); NMR H¹ (300 MHz, CDCl₃, ppm) δ: 7.95 (s, 1H), 7.75 (d, 1H), 7.4 (m, 2H), 6.9 (m, 1H), 5.85 (d, 1H), 5.25 (m, 1H), 4.35 (t, 2H), 3.75 (t, 2H), 3.3 (s, 3H).

### Intermediate 11: 1-methyl-6-formyl-benzimidazole

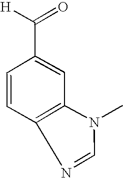

To a solution of intermediate 8 (1.8 g, 11.4 mmol) in dioxane (100 ml) and water (14 ml) was added osmium tetroxide (solution 2.5% in water, 6 ml) and the mixture was stirred for 5 minutes at room temperature. Then sodium periodate (5.1 g, 23.9 mmol) was added and the mixture was stirred at room temperature for 3 hours and then poured into water. The aqueous phase was extracted with CH₂Cl₂, the organic phase was dried over Na₂SO₄ and concentrated under reduced pressure. The title compound was obtained as a cream powder (1 g, 55%); m.p. 132-134° C.

### Intermediate 12: 1-ethyl-6-formyl-benzimidazole

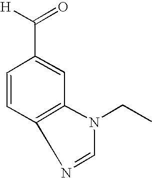

Intermediate 9 (1.5 g, 8.8 mmol) was reacted as described for intermediate 11 to afford the titled compound as an oil (1.3 g, 85.67%); NMR H¹ (300 MHz, CDCl₃, ppm) δ: 10.1 (s, 1H), 8.15 (s, 1H), 8.05 (s, 1H), 7.95 (d, 1H), 7.85 d, 1H), 4.3 (q, 2H), 1.6 (t, 3H).

### Intermediate 13: 1-(2-methoxyethyl)-6-formyl-benzimidazole

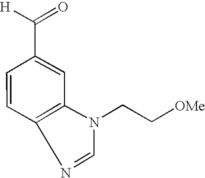

Intermediate 10 (3.5 g, 17.33 mmol) was reacted as described for intermediate 11 to afford the titled compound as an oil (1.3 g, 36.78%); NMR H¹ (300 MHz, CDCl₃, ppm) δ: 10.05 (s, 1H), 8.15 (s, 1H), 8 (s, 1H), 7.9 (d, 1H), 7.8 (d, 1H), 4.4 (t, 2H), 3.7 (t, 2H), 3.3 (s, 3H).

### Intermediate 14: ethyl[(4-formyl-2-nitrophenyl)oxy]acetate

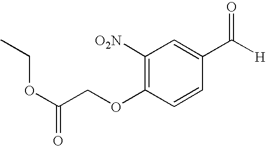

To a solution of 4-hydroxy-3-nitro-benzaldehyde (8 g, 48 mmol) in acetonitrile (200 ml) were added potassium carbonate (10 g, 72 mmol) and ethyl bromoacetate (8 ml, 72 mmol) and the mixture was heated under reflux for 24 hours and then poured into water. After extraction with CH₂Cl₂, the organic phase was dried over Na₂SO₄ and concentrated under reduced pressure. The residue was purified by chromatography on silica gel (CH₂Cl₂). The title compound was obtained as an oil which crystallised on standing (3 g, 24.75%); NMR H¹ (300 MHz, CDCl₃, ppm) δ: 9.95 (s, 1H), 8.35 (s, 1H), 8.05 (d, 1H), 7.1 (d, 1H), 4.85 (s, 2H), 4.25 (q, 2H), 1.25 (t, 3H).

### Intermediate 15: 6-formyl-4H-benzo[1,4]oxazin-3-one

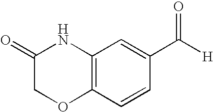

To a solution of intermediate 14 (3 g, 11.8 mmol) in acetic acid (200 ml) was added portionwise iron (6.63 g, 118 mmol) and the mixture was heated at 60° C. for 2 hours and then poured into water. The mixture was basified with a NaOH solution and extracted with CH₂Cl₂. The organic phase was dried over Na₂SO₄, filtered through celite and concentrated under reduced pressure. The title compound was obtained as a amorphous solid (1.8 g, 85.76%); [APCI MS] m/z 176 MH⁻.

### Intermediate 16: 4-methyl-6-formyl-4H-benzo[1,4]oxazin-3-one

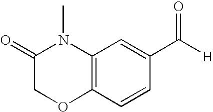

To a solution of intermediate 15 (1 g, 5.6 mmol) in acetone (60 ml) were added potassium carbonate (1.17 g, 8.5 mmol) and methyl iodide (0.53 ml, 8.5 mmol) and the mixture was heated at 60° C. for 16 hours and then poured into water. After extraction with CH₂Cl₂, the organic phase was dried over Na₂SO₄ and concentrated under reduced pressure. The title compound was obtained as a pale yellow oil (1 g, 97.25%); NMR H¹ (300 MHz, CDCl₃, ppm) δ: 9.9 (s, 1H), 7.5 (m, 2H), 7.05 (d, 1H), 4.7 (s, 2H), 3.4 (s, 3H).

### Intermediate 17: 4-ethyl-6-formyl-4H-benzo[1,4]oxazin-3-one

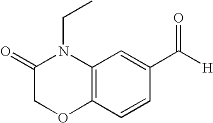

Intermediate 15 (0.8 g, 4.5 mmol) was reacted as described for intermediate 16, replacing methyl iodide by ethyl iodide, to afford the title compound as a yellow oil (0.75 g, 81%); NMR H¹ (300 MHz, CDCl₃, ppm) δ: 9.9 (s, 1H), 7.5 (m, 2H), 7.1 (d, 1H), 4.7 (s, 2H), 4.05 (q, 2H), 1.3 (t, 3H).

### Intermediate 18: [(6-Methylpyridin-2-yl)-phenylamino-methyl]-phosphonic acid diphenylester

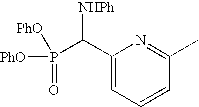

To a solution of 6-methyl-2-pyridinecarboxaldehyde (10 g, 82.55 mmol) in iPrOH (200 ml) were added aniline (1.2 eq, 9.21 g, 99 mmol) and diphenylphosphite (26.85 ml, 115 mmol). The reaction mixture crystallised after 30 min at room temperature. iPrOH (300 ml) was added and the mixture was stirred for 2 h. The resulting solid was filtered and dried to give the title compound as a white solid (40 g, 99.53%); m.p. 110-112° C.

### Intermediate 19: [(3-hydroxy-4-nitro-phenyl)-(phenylamino)-methyl]-phosphonic acid diphenylester

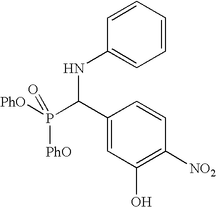

3-Hydroxy-4-nitro-benzaldehyde (11.5 g, 69 mmol) was reacted with aniline and diphenylphosphite as described for Intermediate 18, to afford the title compound as a yellow solid (28 g, 85.4%); [APCI MS] m/z: 475 MH⁻.

### Intermediate 20: 2-[3-hydroxy-4-nitrophenyl]-1-[6-methylpyridin-2-yl]-ethanone

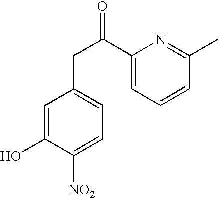

To a solution of 3-hydroxy-4-nitro-benzaldehyde (15 g, 90 mmol) and intermediate 18 (38.82 g, 90 mmol) in THF (200 ml) and iPrOH (200 ml) was added cesium carbonate (88 g, 27 mmol) and the mixture was stirred at room temperature overnight. The mixture was acidified to pH3 by addition of 4N HCl, and allowed to stir at room temperature for 2 hours and then poured in water. After neutralisation with 1N NaOH, the aqueous phase was extracted with CH₂Cl₂, the organic phase was dried over Na₂SO₄ and concentrated under reduced pressure. The residue was purified by chromatography on silica gel eluting with CH₂Cl₂/MeOH (8/2). The title compound was obtained as an orange solid (15 g, 61.4%); m.p. 128-130° C.

### Intermediate 21: 1-[3-hydroxy-4-nitrophenyl]-2-[6-methylpyridin-2-yl]-ethanone

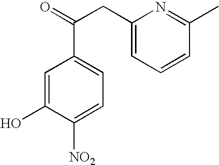

Intermediate 19 (13 g, 27 mmol) and 6-methyl-pyridine-2-carboxaldehyde (TCI Jp, 3 g, 24.8 mmol) were reacted as described for intermediate 20 to afford, the title compound as a solid (5 g, 74.14%); [APCI MS] m/z: 273 MH⁺.

### Intermediate 22: 2-[1-ethyl-benzimidazol-6-yl]-1-[6-methylpyridin-2-yl]-ethanone

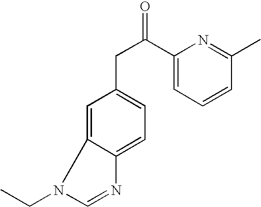

Intermediate 12 (1.3 g, 7.5 mmol) and intermediate 18 (3.86 g, 9 mmol) were reacted as described for intermediate 20 to afford the title compound as a brown oil (1 g, 47.97%); NMR H¹ (300 MHz, CDCl₃, ppm) δ: 7.9 (s, 1H), 8 (m, 1H), 7.85 (m, 1H), 7.55 (s, 1H), 7.35 (m, 1H), 7.2 (m, 1H), 6.9 (m, 1H), 4.7 (s, 2H), 4.25 (q, 2H), 2.65 (s, 3H), 1.55 (t, 3H).

### Intermediate 23: 2-[1-(2-methoxyethyl)-benzimidazol-6-yl]-1-[6-methylpyridin-2-yl]-ethanone

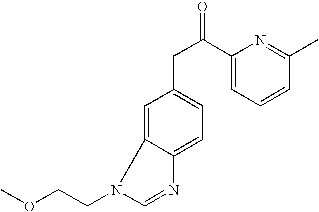

Intermediate 13 (1.3 g, 6.4 mmol) and intermediate 18 (3.29 g, 7.6 mmol) were reacted as described for intermediate 20 to afford the title compound as a brown oil (1.2 g, 60.94%); NMR H¹ (300 MHz, CDCl₃, ppm) δ: 8.00 (s, 1H), 7.9 (m, 1H), 7.75 (m, 2H), 7.45 (s, 1H), 7.3 (m, 1H), 7.1 (m, 1H), 4.7 (s, 2H), 4.3 (t, 2H), 3.7 (t, 2H), 3.3 (s, 3H), 2.6 (s, 3H).

### Intermediate 24: 2-[4-methyl-4H-benzo[1,4]oxazin-3-one-6-yl]-1-[6-methylpyridin-2-yl]-ethanone

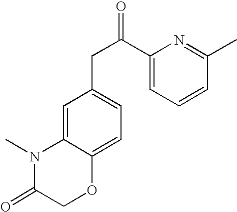

Intermediate 16 (1 g, 5.5 mmol) and intermediate 18 (2.84 g, 6.6 mmol) were reacted as described for intermediate 20 to afford the title compound as a brown oil (1.3 g, 79.93%); [APCI MS] m/z 297 MH⁺.

### Intermediate 25: 2-[4-ethyl-4H-benzo[1,4]oxazin-3-one-6-yl]-1-[6-methylpyridin-2-yl]-ethanone

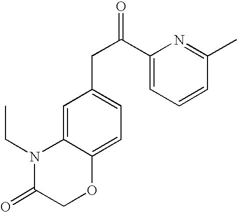

Intermediate 26 (0.75 g, 3.66 mmol) and intermediate 18 (1.89 g, 4.39 mmol) were reacted as described for intermediate 20 to afford the title compound as a red oil (0.5 g, 44.1%); [APCI MS] m/z 311 MH⁺.

### Intermediate 26: 2-[3-(trifluopromethanesulfonate)-4-nitrophenyl]-1-[6-methylpyridin-2-yl]-ethanone

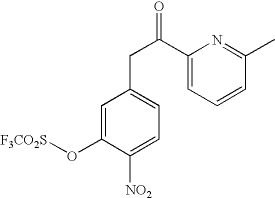

To a solution of intermediate 20 (15 g, 55 mmol) in CH₂Cl₂ (400 ml) were added aniline-N-bis (trifluoromethanesulfonate) (30 g, 83 mmol) and triethylamine (11.5 ml, 83 mmol) and the mixture was stirred at room temperature for 24 hours and then poured into water. After extraction with CH₂Cl₂, the organic phase was dried over Na₂SO₄ and concentrated under reduced pressure. After crystallisation from acetonitrile, the title compound was obtained as yellow crystals (21 g, 94.25%); m.p. 118-120° C.

### Intermediate 27: 2-[3-(methylamino)₄-nitrophenyl]-1-[6-methylpyridin-2-yl]-ethanone

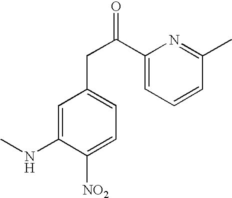

A solution of intermediate 26 (21 g, 52 mmol) in methylamine (2M in THF, 200 ml) was heated at 150° C. in a schlenk tube for 2 hours and then cooled and poured into water. After extraction with CH₂Cl₂, the organic phase was dried over Na₂SO₄, and concentrated under reduced pressure. The residue was purified by chromatography on silic agel eluting with CH₂Cl₂/cyclohexane (8/2). The title compound was obtained as an orange solid (13 g, 87.75%); m.p. 100-102° C.

### Intermediate 28: 5-(3-methylamino-4-nitro-phenyl)-4-(6-methylpyridin-2-yl)-1,3-thiazol-2-amine

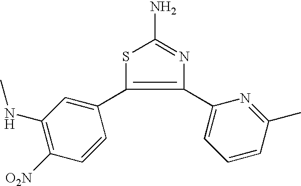

To a solution of Intermediate 27 (13 g, 46 mmol) in CH₂Cl₂ (200 ml) and ethanol (20 ml) was added polymer-supported pyridinium perbromide (10 g) and the suspension was stirred at room temperature for 4 hours. The resin was removed by filtration, and the filtrate added directly to thiourea (5.2 g, 69 mmol) washing the resin through several times with ethanol. The filtrate was heated under reflux for 14 hours, allowed to cool and concentrated under reduced pressure. The residue was basified with aqueous NaOH and extracted with CH₂Cl₂. The organic phase was washed with water, dried over Na₂SO₄ and concentrated under reduced pressure. Chromatography on silica gel (toluene/isopropylamine, 812), gave the title compound as an orange solid (15 g, 96.43%); m.p. 100-102° C.

### Intermediate 29: 4-(3-hydroxy-4-nitro-phenyl)-5-(6-methylpyridin-2-yl)-1,3-thiazol-2-amine

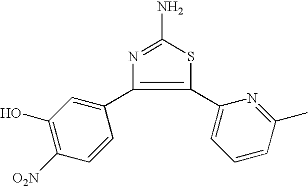

Intermediate 21 (1 g, 3.67 mmol) was reacted as described for intermediate 28, to afford the title compound as a solid (1 g, 82.93%); m.p. 260-262° C.; [APCI MS] m/z=327 MH⁻.

### Intermediate 30: 1-(Benzo[1,3]dioxol-5-yl)-2-(6-methylpyridin-2-yl)-ethanone

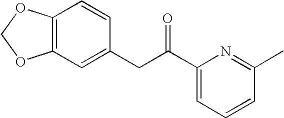

2,6-Lutidine (4.66 g; 43 mmol) and ethyl 1,3-benzodioxole-5-carboxylate (2 g, 10.9 mmol) were dissolved in dry THF (100 ml) under nitrogen and the solution cooled to −20° C. A solution of NaHMDS (2M in THF, 6 ml, 12 mmol) was added at below −20° C. and the mixture stirred for 12 h at −20° C. and then at room temperature overnight. The reaction mixture was concentrated and the resulting residue poured into water and extracted with CH₂Cl₂. The organic phase was washed with a saturated solution of ammonium chloride, washed with a saturated solution of NaCl, dried over Na₂SO₄ and concentrated under reduced pressure. Chromatography on silica gel (CH₂Cl₂/MeOH, 95/5) gave the title compound as a yellow oil (2.2 g, 79%); [APCI MS] m/z=256 MH⁺.

### Intermediate 31: 1-(Benzodioxan-6-yl)-2-(pyridin-2-yl)-ethanone

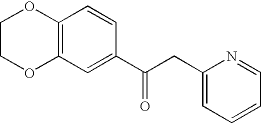

1,4-Benzodioxan-6-carboxylic acid methyl ester was reacted with 2-methylpyridine as described for intermediate 30, to afford the title compound as a yellow oil (1.8 g, 70.6%); [GC MS] m/z=255.

### Intermediate 32: 1-(Quinolin-6-yl)-2-(pyridin-2-yl)-ethanone

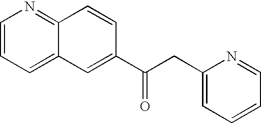

6-Quinolinecarboxylic acid, methyl ester was reacted with 2-methylpyridine as described for intermediate 30, to afford the title compound as a yellow solid (1.34 g, 49.44%); m.p. 104° C.; [APCI MS] m/z=249 MH⁺.

### Intermediate 33: 1-(1-Methylbenzotriazol-6-yl)-2-(6-methylpyridin-2-yl)-ethanone

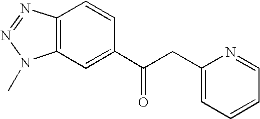

1-Methylbenzotriazole-6-carboxylic acid, methyl ester (prepared as described in patent EP 1247810) was reacted with 2-methylpyridine as described for intermediate 30, to afford the title compound as a yellow solid (0.6 g, 45.74%); [GC MS] m/z=252.

### Intermediate 34: diphenyl [(1-methyl-1H-benzimidazol-6-yl)(phenylamino)methyl]phosphonate

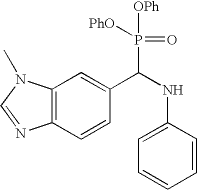

Intermediate 11 (2.2 g, 13.75 mmol) was reacted with aniline and diphenylphosphite as described for Intermediate 18, to afford the title compound as an off-white powder (5.5 g, 85%); [APCI MS] m/z=470 MH⁺.

### Intermediate 35: 1-(1-methyl-1H-benzimidazol-6-yl)-2-(6-methyl-2-pyridinyl)ethanone

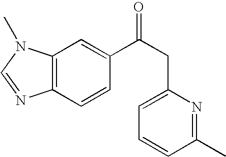

Intermediate 34 (5.5 g, 11.7 mmol) and 6-methyl-2-pyridine carboxaldehyde (1.56 g, 25.8 mmol) were reacted as described for intermediate 20 to afford the title compound as an orange oil (1 g, 32%); [APCI MS] m/z=266 MH⁺.

### EXAMPLES

### Example 1

### 5-(1-methyl-benzimidazol-6-yl)-4-(6-methylpyridin-2-yl)-1,3-thiazol-2-amine

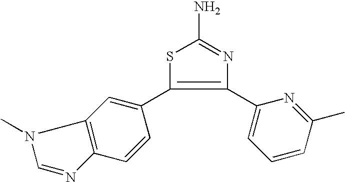

To a solution of intermediate 28 (15 g, 44 mmol) in a mixture of CH₂Cl₂ (300 ml) and acetic acid (80 ml) was added portionwise iron (24.8 g, 440 mmol) under vigorous stirring, and the mixture was stirred at room temperature for 16 hours. After filtration of the reaction mixture through celite, trimethylorthoformate (6.25 ml, 57 mmol) was added to the filtrate and the mixture was heated at 40° C. for 2 hours and then cooled. The mixture was neutralised by addition of 2N NaOH, then filtered through celite and the precipitate washed with water. The filtrate was extracted with CH₂Cl₂, the organic phase dried over Na₂SO₄ and concentrated under reduced pressure. The residue was purified by chromatography on silica gel, eluting with toluene/isopropylamine (8/2). After trituration with pentane, the title compound was obtained as a pale yellow solid (1.5 g, 10.62%); m.p. 244-246° C.; [APCI MS] m/z=322 MH⁺.

### Example 2

### 4-(benzoxazol-6-yl)-5-(6-methyl-pyridin-2-yl)-1,3-thiazol-2-amine

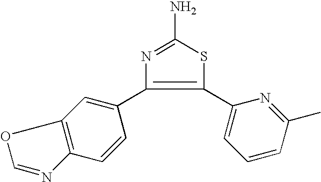

To a solution of intermediate 29 (1 g, 3 mmol) in acetic acid (100 ml) was added portionwise iron (1.68 g, 30 mmol) under vigorous stirring, and the mixture was heated at 60° C. for 3 hours and then poured into water. The mixture was neutralised with aqueous NaOH and extracted with CH₂Cl₂. The organic phase was dried over Na₂SO₄ and concentrated under reduced pressure. The resulting oil was dissolved in EtOH (200 ml) and ethyl formimidate hydrochloride (0.5 g, 4.6 mmol) was added. The mixture was heated under reflux overnight and then concentrated under reduced pressure. The residue was purified by chromatography on silicagel (CH₂Cl₂/MeOH, 9/1). After trituration with pentane, the title compound was obtained as a cream solid, (0.11 g, 11.7%); m.p. 188-190° C.; [APCI MS] m/z=309 MH⁺.

### Example 3

### 5-(1-ethyl-benzimidazol-6-yl)-4-(6-methylpyridin-2-yl)-1,3-thiazol-2-amine

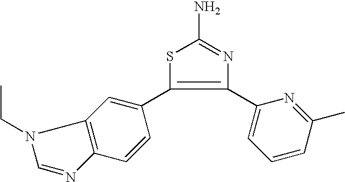

To a solution of Intermediate 22 (0.5 g, 1.8 mmol) in CH₂Cl₂ (80 ml) was added polymer-supported pyridinium perbromide (1.8 g) and the suspension was stirred at room temperature for 4 hours. The resin was removed by filtration, with the filtrate being added directly to thiourea (0.206 g, 2.7 mmol) and the resin washed several times with ethanol. The filtrate was heated under reflux overnight, allowed to cool and concentrated under reduced pressure. The residue was basified with aqueous NaOH, extracted with CH₂Cl₂ and this phase washed with water. The organic phase was dried over Na₂SO₄, and concentrated under reduced pressure. After crystallisation from ethanol, the title compound was obtained as cream crystals (0.25 g, 41.49%); m.p, 218-220° C.; [APCI MS] m/z=336 MH⁺.

### Example 4

### 5-(1-(2-methoxyethyl)-benzimidazol-6-yl)-4-(6-methylpyridin-2-yl)-1,3-thiazol-2-amine

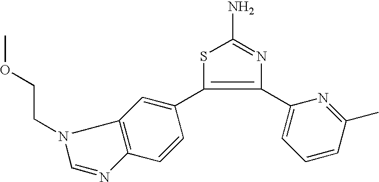

Intermediate 23 (0.6 g, 1.9 mmol) was reacted as described for example 3, to afford after crystallisation from acetonitrile, the title compound as cream crystals (0.12 g, 17.3%); m.p. 200-202° C.; [APCI MS] m/z=366 MH⁺.

### Example 5

### 5-[4-methyl-4H-benzo[1,4]oxazin-3-one-6-yl]-4-(6-methylpyridin-2-yl)-1,3-thiazol-2-amine

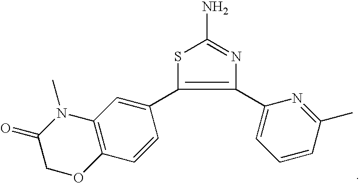

To a solution of Intermediate 24 (1.3 g, 4.4 mmol) In CH₂Cl₂ (50 ml) was added polymer-supported pyridinium perbromide (2 g) and the suspension was stirred at room temperature for 3 hours. The resin was removed by filtration, then the filtrate was directly added to thiourea (0.34 g, 4.4 mmol) and the resin washed many times with ethanol. The filtrate was heated under reflux for 16 hours, allowed to cool at room temperature and concentrated under reduced pressure. The residue was basified with aqueous NaOH, extracted with CH₂Cl₂ and this phase washed with water. The organic phase was dried over Na₂SO₄, and concentrated under reduced pressure. After chromatography on silica gel (CH₂Cl₂/MeOH, 9/1) and then trituration with diisopropyle oxyde, the title compound was obtained as a cream solid (0.3 g, 19.4%); m.p. 240-242° C.; [APCI MS] m/z=353 MH⁺.

### Example 6

### 5-[4-ethyl-4H-benzo[1,4]oxazin-3-one-6-yl]-4-(6-methylpyridin-2-yl)-1,3-thiazol-2-amine

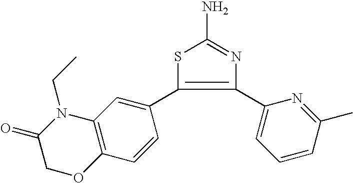

Intermediate 25 (0.5 g, 1.6 mmol) was reacted as described for example 5 to afford after chromatography on silica gel (CH₂Cl₂/MeOH, 9/1) and then trituration with diisopropyl oxide, the title compound as a cream solid (0.09 g, 15.24%); m.p. 228-230° C.; [APCI MS] m/z=367 MH⁺,

### Example 7

### 4-(Benzo[1,3]dioxol-5-yl)-5-(6-methylpyridin-2-yl)-1,3-thiazol-2-amine

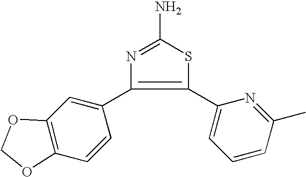

Intermediate 30 (1 g, 3.9 mmol) was reacted as described for example 5 to afford after chromatography on silica gel (CH₂Cl₂/MeOH, 95/5) and then trituration with pentane, the title compound as a yellow powder (0.52 g, 42.87%); m.p. 170-172° C.; [APCI MS] m/z=312 MH⁺.

### Example 8

### 4-(Benzodioxan-6-yl)-5-(Pyridin-2-yl)-1,3-thiazol-2-amine

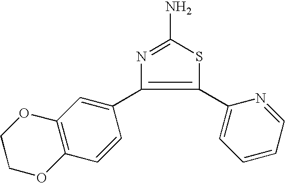

Intermediate 31 (0.51 g, 2 mmol) was reacted as described for example 5 to afford after recrystallisation with EtOH, the title compound as a yellow solid (0.35 g, 56.3%); m.p. 214° C.; [APCI MS] m/z=312 MH⁺.

### Example 9

### 4-(Quinolin-6-yl)-5-(pyridin-2-yl)-1,3-thiazol-2-amine

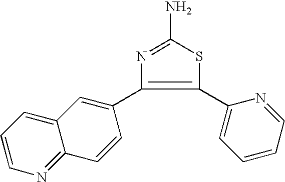

Intermediate 33 (0.7 g, 2.82 mmol) was reacted as described for examples 5 to afford after recrystallisation from iPrOH, the title compound as a yellow solid (0.2 g, 23.9%); m.p. 226° C.; [APCI MS] m/z 305 MH⁺.

### Example 10

### 4-(1-Methyl-benzotriazol-6-yl)-5-(pyridin-2-yl)-1,3-thiazol-2-amine

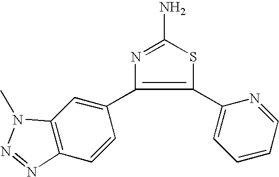

Intermediate 33 (0.8 g, 3.25 mmol) was reacted as described for example 5 to afford after recrystallisation from iPrOH, the title compound as a light beige powder (0.07 g, 30.23%). [APCI MS] m/z=309 MH⁺; TOF MS ES⁺ exact mass calculated for C₁₅H₁₂N₆S (MH+):309.0922. Found: 309.0926.

### Example 11

### 4-(1-Methyl-benzimidazol-6-yl)-5-(6-methylpyridin-2-yl)-1,3-thiazol-2-amine

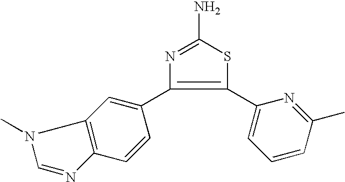

Intermediate 35 (0.5 g, 1.9 mmol) was reacted as described for example 5, to afford, after recrystallisation with ethanol, the title compound as a beige powder (0.15 g, 12%); ¹H NMR (300 MHz; CDCl₃) δ: 7.80 (s, 1H), 7.65 (d, 1H), 7.60 (s, 1H), 7.35 (d, 1H), 7.15 (t, 1H), 6.80 (t, 2H), 5.25 (brs, 2H), 3.75 (s, 3H), 2.45 (s, 3H); TOF MS ES* exact mass calculated for C₁₇H₁₅N₅S (MH+):322.2126. Found: 322.2120.

Biology

The biological activity of the compounds of the invention may be assessed using the following assays:

Assay 1 (Cellular Transcriptional Assay)

The potential for compounds of the invention to inhibit TGF-β signaling may be demonstrated, for example, using the following in vitro assay.

The assay was performed in HepG2 cells stably transfected with the PAI-1 promoter (known to be a strong TGF-β responsive promoter) linked to a luciferase (firefly) reporter gene. The compounds were selected on their ability to inhibit luciferase activity in cells exposed to TGF-β. In addition, cells were transfected with a second luciferase (Renilla) gene which was not driven by a TGF-β responsive promoter and was used as a toxicity control.

96 well microplates were seeded, using a multidrop apparatus, with the stably transfected cell line at a concentration of 35000 cells per well in 200 μl of serum-containing medium. These plates were placed, in a cell incubator.

18 to 24 hours later (Day 2), cell-incubation procedure was launched: Cells were incubated with TGF-β and a candidate compound at concentrations in the range 50 nM to 10 μM (final concentration of DMSO 1%). The final concentration of TGF-β (rhTGFβ-1) used in the test was 1 ng/mL. Cells were incubated with a candidate compound 15-30 mins prior to the addition of TGF-β. The final volume of the test reaction was 150 μl. Each well contained only one candidate compound and its effect on the PAI-1 promoter was monitored.

Columns 11 and 12 were employed as controls. Column 11 contained 8 wells in which the cells were incubated in the presence of TGF-β, without a candidate compound. Column 11 was used to determine the ‘reference TGF-β induced firefly luciferase value’ against which values measured in the test wells (to quantify inhibitory activity) were compared. In wells A12 to D12, cells were grown in medium without TGF-β. The firefly luciferase values obtained from these positions are representative of the ‘basal firefly luciferase activity’. In wells E12 to H12, cells were incubated in the presence of TGF-β and 500 μM CPO (Cyclopentenone, Sigma), a cell toxic compound. The toxicity was revealed by decreased firefly and renilla luciferase activities (around 50% of those obtained in column 11).

12 to 18 hours later (day 3), the luciferase quantification procedure was launched. The following reactions were performed using reagents obtained from a Dual Luciferase Assay Kit (Promega). Cells were washed and lysed with the addition of 10 μl of passive lysis buffer (Promega). Following agitation (15 to 30 mins), luciferase activities of the plates were read in a dual-injector luminometer (BMG lumistar). For this purpose, 50 μl of luciferase assay reagent and 50 μl of ‘Stop & Glo’ buffer were injected sequentially to quantify the activities of both luciferases. Data obtained from the measurements were processed and analysed using suitable software. The mean Luciferase activity value obtained in wells A11 to H11 (Column 11, TGF-β only) was considered to represent 100% and values obtained in wells A12 to D12 (cells in medium alone) gave a basal level (0%). For each of the compounds tested, a concentration response curve was constructed from which an IC₅₀ value was determined graphically.

Assay 2 (Alk5 Fluorescence Polarization Assay)

Kinase inhibitor compounds conjugated to fluorophores, can be used as fluorescent ligands to monitor ATP competitive binding of other compounds to a given kinase. The increase in depolarization of plane polarized light, caused by release of the bound ligand into solution, is measured as a polarization/anisotropy value. This protocol details the use of a rhodamine green-labelled ligand for assays using recombinant GST-ALK5 (residues 198-503).

Assay buffer components: 62.5 mM Hepes pH 7.5 (Sigma H-4034), 1 mM DTT (Sigma D-0632), 12.5 mM MgCl₂ (Sigma M-9272), 1.25 mM CHAPS (Sigma C-3023).

Protocol: Solid compound stocks were dissolved in 100% DMSO to a concentration of 1 mM and transferred into column 1, rows A-H of a 96-well, U bottom, polypropylene plate (Costar #3365) to make a compound plate. The compounds were serially diluted (3-fold in 100% DMSO) across the plate to column 11 to yield 11 concentrations for each test compound. Column 12 contained only DMSO. A Rapidplate™-96 was used to transfer 1 fl of sample from each well into a 96-well, black, U-bottom, non-treated plate (Costar #3792) to create an assay plate.

ALK5 was added to assay buffer containing the above components and 1 nM of the rhodamine green-labelled ligand so that the final ALK5 concentration was 10 nM based on active site titration of the enzyme. The enzyme/ligand reagent (39 μl) was added to each well of the previously prepared assay plates. A control compound (1 μl) was added to column 12, rows E-H for the low control values. The plates were read immediately on a UL Acquest fluorescence reader (Molecular Devices, serial number AQ1048) with excitation, emission, and dichroic filters of 485 nm, 530 nm, and 505 nm, respectively. The fluorescence polarization for each well was calculated by the Acquest reader and then imported into curve fitting software for construction of concentration response curves. The normalized response was determined relative to the high controls (1 μl DMSO in column 12, rows 4-D) and the low controls (1 μl of control compound in column 12, rows E-H). An IC₅₀ value was then calculated for each compound

Using the above assays all Examples of the invention show ALK5 receptor modulator activity (having IC₅₀ values in the range of 1 to 100 nM) and TGF-β cellular activity (having IC₅₀ values in the range of 0.001 to 10 μM).

5-(1-Methyl-benzimidazol-6-yl)-4-(6-methylpyridin-2-yl)-1,3-thiazol-2-amine (Example 1) showed an ALK5 receptor modulator activity of 16 nM and TGF-D cellular activity of 11 nM.

5-[4-Ethyl-4H-benzo[1,4]oxazin-3-one-6-yl]4-(6-methylpyridin-2-yl)-1,3-thiazol-2-amine (Example 6) showed an ALK5 receptor modulator activity of 27 nM and TGF-62 cellular activity of 104 nM.

What is claimed is:

1. A compound of formula (I), a pharmaceutically acceptable salt, solvate or derivative thereof: 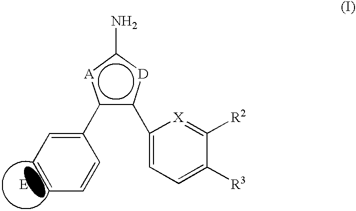
wherein
either A is S and D is N; or A is N and D is S;
ring E is a saturated, unsaturated or aromatic 5 or 6-membered heterocycle which heterocycle in addition to carbon contains one or more ring-heteroatoms independently selected from nitrogen and oxygen, wherein the heterocycle is optionally substituted on any nitrogen atom where appropriate by one or more groups R^{Ea} independently selected from C₁₋₆alkyl and C₁₋₆alkoxyC₁₋₆alkyl and is optionally substituted on any carbon atom where appropriate by one or more groups R^{Eb} independently selected from oxo, C₁₋₆alkyl, C₁₋₆alkoxyC₁₋₆alkyl, C₁₋₆alkoxy and halo;
X is N or CH;
R² is hydrogen, C₁₋₆alkyl, halo, cyano or perfluoroC₁₋₆alkyl; and
R³ is hydrogen or halo.

2. A compound according to claim 1 where the benzofused ring system including E is selected from the list: benzimidazol-6-yl, benzoxazol-6-yl, benzoxazol-5-yl, 4H-benzo[1,4]oxazin-3-one-6-yl, benzo[1,3]dioxol-5-yl, benzodioxan-6-yl, quinolin-6-yl and benzotriazol-6-yl.

3. A compound according to claim 1 where X is N.

4. A compound according to claim 1 where R² is hydrogen, C₁₋₆alkyl, chloro or fluoro.

5. A compound according to claim 4 where R² is hydrogen, methyl, chloro or fluoro.

6. A compound according to claim 5 where R² is methyl.

7. A compound according to claim 1 where R³ is hydrogen.

8. A compound according to claim 1 wherein, when X is N, R² is methyl.

9. A compound according to claim 8 wherein when X is N and R² is methyl, R³ is H.

10. A compound according to claim 1 selected from:
5-(1-methyl-benzimidazol-6-yl)-4-(6-methylpyridin-2-yl)-1,3-thiazol-2-amine;
4-(benzoxazol-6-yl)-5-(6-methyl-pyridin-2-yl)-1,3-thiazol-2-amine;
5-(1-ethyl-benzimidazol-6-yl)-4-(6-methylpyridin-2-yl)-1,3-thiazol-2-amine;
5-(1-(2-methoxyethyl)-benzimidazol-6-yl)-4-(6-methylpyridin-2-yl)-1,3-thiazol-2-amine;
5-[4-methyl-4H-benzo[1,4]oxazin-3-one-6-yl]-4-(6-methylpyridin-2-yl)-1,3-thiazol-2-amine;
5-[4-ethyl-4H-benzo[1,4]oxazin-3-one-6-yl]-4-(6-methylpyridin-2-yl)-1,3-thiazol-2-amine;
4-(benzo[1,3]dioxol-5-yl)-5-(6-methylpyridin-2-yl)-1,3-thiazol-2-amine;
4-(benzodioxan-6-yl)-5-(pyridin-2-yl)-1,3-thiazol-2-amine;
4-(quinolin-6-yl)-5-(pyridin-2-yl)-1,3-thiazol-2-amine;
4-(1-methyl-benzotriazol-6-yl)-5-(pyridin-2-yl)-1,3-thiazol-2-amine;
4-(1-methyl-benzimidazol-6-yl)-5-(6-methylpyridin-2-yl)-1,3-thiazol-2-amine; and
pharmaceutically acceptable salts, solvates and derivatives thereof.

11-13. (canceled)

14. A method of treatment or prophylaxis of a disorder selected from chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photoaging, in mammals, which comprises administration to the mammal in need of such treatment, an effective amount of a compound of formula (I) as defined in claim 1.

15. A pharmaceutical composition comprising a compound of formula (I) as claimed in claim 1 and a pharmaceutically acceptable diluent or carrier.

16. A combination of a compound of formula (I) as claimed in claim 1 with an ACE inhibitor or an angiotensin II receptor antagonist.
